(19) United States
(12) Patent Application Publication
(10) Pub. No.: US 2011/0059149 A1
(43) Pub. Date: Mar. 10, 2011

(54) Process for depositing calcium phosphate therapeutic coatings with different release rates and a prosthesis coated via the process

(76) Inventors: Marisa A. Little, Bedford, MA (US); Nader M. Kalkhoran, Tewksbury, MA (US); Arash Aslani, Acton, MA (US); Eric J. Tobin, North Andover, MA (US); Jason E. Burns, Cambridge, MA (US)

(21) Appl. No.: 12/806,608

(22) Filed: Aug. 17, 2010

Related U.S. Application Data
(63) Continuation-in-part of application No. 12/214,037, filed on Jun. 16, 2008.
(60) Provisional application No. 61/274,498, filed on Aug. 18, 2009.

(51) Int. Cl. A61F 2/00; B05D 1/36; C23C 14/46

(52) U.S. Cl. 424/423; 427/2.27; 204/192.11

(57) ABSTRACT

A method of coating a substrate including loading a calcium phosphate substance at a first crystallinity with a therapeutic agent; depositing the loaded calcium phosphate substance at the first crystallinity onto a least a portion of the substance; loading a calcium phosphate substance at a second, lower, crystallinity with a therapeutic agent; and depositing the loaded calcium phosphate substance at the second crystallinity onto the deposited loaded calcium phosphate substance at the first crystallinity to control and sustain a long-term osseointegration response and to control the release rate of the therapeutic substance from the calcium phosphate substance.

### RELATED APPLICATIONS

This application is a continuation-in-part of prior U.S. patent application Ser. No. 12/214,037 filed Jun. 16, 2008 and claims the benefit of and priority to U.S. Provisional Patent Application Ser. No. 61/274,498 filed Aug. 18, 2009, and each application is incorporated herein by reference.

### FIELD OF THE INVENTION

This subject invention relates to implants, coatings for implants, and therapeutic agents such as bone morphogenic proteins.

### BACKGROUND OF THE INVENTION

Implants made of titanium, cobalt chrome, and other materials are often coated with one or more layers of a calcium phosphate material such as hydroxyapatite to promote bony fixation (biointegration) wherein bone grows onto and/or into the surface of the implant. It is also known to add a therapeutic agent to the implant such as a bone morphogenic protein (e.g., BMP, or GDF-5) to promote bone growth.

U.S. Pat. No. 6,821,528, incorporated herein by this reference, discloses a process wherein calcium phosphate in the form of hydroxyapatite is precipitated from a solution to coat the implant. Next, the coated implant is dried, sterilized, and packaged. Just before implantation, the coated implant is immersed in a morphogenic protein solution.

In such a process, the release rate of the therapeutic agent is difficult to control. Also, the surgeon is required to immerse the implant in the morphogenic protein solution.

It is also known to adhere a hydroxyapatite layer to the surface of an implant by plasma thermal spraying. See U.S. Pat. No. 5,934,287 incorporated herein by this reference. That patent discloses a different process wherein amorphous calcium phosphate particles are sandblasted onto an implant to form a coating. A therapeutic agent is not present and thus bone growth may not be adequately promoted.

According to U.S. Pat. No. 6,949,251, also incorporated herein by this reference, an implant comprises a porous β-TCP matrix and a bioactive agent such as a bone morphogenic protein preferably encapsulated in a biodegradable agent such as a polymer. Also, a composition including β-TCP and a bioactive agent can be disposed on the surface of an implant. Polymers used in controlled delivery systems have been known to cause complications.

U.S. Patent Publication No. 2006/0088565, also incorporated herein by this reference, discloses a pharmaceutical composition for bone repair wherein a calcium phosphate carrier is coated with a protein such as BMP.

U.S. Pat. No. 6,261,322 (also incorporated herein by this reference) discloses coating an implant with a biocompatible coating which may include a calcium phosphate. Physical or chemical vapor deposition is used to coat the implant. The implant may have multiple layers and/or nanolayers.

U.S. Pat. No. 6,969,474 (incorporated herein by this reference) discloses acid etching the surface of an implant and depositing particles of a bone growth enhancing material such as bone morphogenic proteins or hydroxyapatite onto the etched surface of the implant.

U.S. Patent Publication Nos. 2006/0210494 and 2009/0304761, incorporated herein by this reference, disclose functionally graded coatings which are generally crystalline at the implant interface and decreasing in crystallinity toward the outer layer of the coating. Dual ion beam sputtering is used to deposit the coating.

### SUMMARY OF THE INVENTION

One aspect of this invention is to provide a new method of coating an implant. The implant, in one example, promotes osteogenesis, osteoconduction, and osteoinduction. A therapeutic agent such as a BMP is included in a coating and the coating is deposited in a way in which the efficacy of the therapeutic agent is not adversely affected. The release rate of the therapeutic agent from the coating is tailored once applied to the implant and implanted into a patient.

The invention results, at least in part, from the realization that by loading calcium phosphate with a therapeutic agent such as a bone morphogenic protein and controlling the crystallinity of the calcium phosphate, the release rate of the therapeutic agent can be tailored and also that by using a Accelerated Particle Deposition (APD) process to coat the implant with the loaded calcium phosphate, the efficacy of the therapeutic agent is not adversely affected.

The subject invention, however, in other embodiments, need not achieve all these objectives and the claims hereof should not be limited to structures or methods capable of achieving these objectives.

This subject invention features a method of coating a substrate and a product made by the method. The method includes controlling the crystallinity of a calcium phosphate substance in a coating material, loading the calcium phosphate substance with a therapeutic agent, and depositing the loaded calcium phosphate onto at least a portion of the substrate. The calcium phosphate substance may be amorphous calcium phosphate, fluorapatite, hydroxyapatite, tetracalcium phosphate, tricalcium phosphate-alpha, tricalcium phosphate-beta, biphasic calcium phosphate, silica calcium phosphate, and/or multiphasic calcium phosphate. The calcium phosphate substance typically has a grain size between 10 nm and 10 microns.

Controlling the crystallinity may include choosing nanocrystalline calcium phosphate particles, choosing microcrystalline calcium phosphate particles, forming powder particles of loaded calcium phosphate wherein a certain percentage of the calcium phosphate is amorphous and a certain percentage of the calcium phosphate is crystalline in structure, or forming a certain percentage of powder particles of loaded calcium phosphate having one characteristic and mixing the same with a certain percentage of powder particles of loaded calcium phosphate having a different characteristic.

Depositing may include employing a gas accelerated particle deposition process, electrophoretic deposition, or a physical vapor deposition process, such as ion beam sputtering or ion beam assisted deposition. Particles of loaded calcium phosphate between 0.001 to 200 μm may be entrained in a gas jet at 50 to 220 psi and directed to the surface of the substrate at a distance of 0.5 to 2 inches.

Loading may include mixing a therapeutic substance in solution with calcium phosphate in powder form. Calcium phosphate can be precipitated from a solution including the therapeutic substance. The loaded calcium phosphate can be deposited to a thickness of between 0.1-30 μm. In one example, the therapeutic substance is a bone morphogenic compound and/or an antibiotic.

The subject invention also features an implant with a coating on at least a portion of its surface, the coating comprising particles of calcium phosphate of a predetermined crystallinity loaded with a therapeutic agent imbedded into the implant. The therapeutic compound is released from the coating in a controlled, predetermined manner.

The calcium phosphate substance may be amorphous calcium phosphate, fluorapatite, hydroxyapatite, tetracalcium phosphate, tricalcium phosphate-alpha, tricalcium phosphate-beta, biphasic calcium phosphate, silica calcium phosphate, and/or multiphasic calcium phosphate. The calcium phosphate grain size may be between 10 nm and 10 microns. The calcium phosphate may include nanocrystalline calcium phosphate particles. The calcium phosphate may also include microcrystalline calcium phosphate particles. A certain percentage of the calcium phosphate may be amorphous and a certain percentage of the calcium phosphate may be crystalline in structure. A certain percentage of powder particles of loaded calcium phosphate may have one characteristic and can be mixed with a certain percentage of powder particles of loaded calcium phosphate having a different characteristic. A gas accelerated particle deposition process, electrophoretic deposition, or a physical vapor deposition process, such as ion beam sputtering or ion beam assisted deposition can be used to coat the particles onto the implant. Particles of loaded calcium phosphate between 0.001 to 200 μm may be entrained in a gas jet at 50 to 220 psi and directed to the surface of an implant at a distance of 0.5 to 2 inches.

A therapeutic substance in solution may be mixed with calcium phosphate in powder form. Calcium phosphate can be precipitated from a solution including the therapeutic substance. A typical coating has a thickness of between 0.1-30 μm. The therapeutic substance may be a bone morphogenic compound and/or an antibiotic.

A method for coating a substrate may include loading a calcium phosphate substrate at a first crystallinity of between 50% and 100% with a therapeutic agent of up to 50% by weight, and loading a calcium phosphate substance at a second crystallinity of between amorphous or near amorphous to 50% crystallinity with a therapeutic agent of up to 50% volume by weight. The method may further use a gas accelerated particle deposition process to deposit the loaded calcium phosphate substance at the first higher crystallinity onto at least a portion of the substrate to form a firm film having a first thickness, and depositing the loaded calcium phosphate substance at the second lower crystallinity onto the first film to form a second film thereon having a second thickness thinner than the thickness of the first film.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a block diagram showing the primary components associated with a subsystem used to deposit a calcium phosphate coating onto a substrate in accordance with the subject invention;

FIG. 2 is a SEM cross-sectional view of a calcium phosphate coating applied to a titanium substrate in accordance with the subject invention;

FIG. 3 is another SEM cross-sectional view of a calcium phosphate coating applied to a titanium substrate in accordance with the subject invention;

FIG. 4 is a profilometry scan of an uncoated substrate;

FIG. 5 is a profilometry scan of a substrate coated with calcium phosphate in accordance with the subject invention;

FIG. 6 is a graph showing the tensile strength for various calcium phosphate coatings in accordance with the subject invention;

FIG. 7 is a graph showing the shear strength of a variety of calcium phosphate coatings;

FIG. 8 is a graph showing the release profile of a therapeutic agent in micrograms over time when the coating includes amorphous calcium phosphate in accordance with the subject invention;

FIG. 9 is a graph showing the release profile of a therapeutic agent in micrograms over time when the coating includes microcrystalline calcium phosphate in accordance with the subject invention;

FIG. 10 is a graph showing the percent change in the osteoblast number when cultured with supernatant of BMP-2 released from a nano-amorphous calcium phosphate coating over 21 days;

FIG. 11 is a highly schematic cross-sectional view of an example of a calcium phosphate particle loaded with a therapeutic agent in accordance with the subject invention;

FIG. 12 is a schematic depiction of functionally graded HA thin film. The film consists of an amorphous/small-grained mixture region at the surface that rapidly resorbs and stimulates initial bone growth, and larger-grained crystalline, more slowly resorbing regions near the coating/substrate interface, which will provide long-term coating stability and device fixation;

FIG. 13 is a transmission electron micrograph of a deposited crystallographically graded metallic thin film;

FIGS. 14A and 14B show XRD spectra from as-deposited and annealed IBAD Ca—P coatings, respectively;

FIG. 15 shows the mean push-out strength for HA coated Ti cylinders implanted in rat tibiae. Highest push-out strengths at both 3 and 9 weeks were observed for the intermediate crystallinity films: 50% and 70% crystalline;

FIG. 16 shows results of dissolution studies, which monitored calcium and phosphorus release from each of four coatings of varying crystallinity (HA1=30% crystallinity, HA2=50%, HA3=70%, HA4=90%). Dissolution rates for both elements increase with decreasing coating crystallinity;

FIG. 17 shows the mean push-out strength for uncoated and CaP coated Ti cylinders implanted in rat tibiae after 9 weeks. The highest push-out strength was observed for the multi-layer films. (Note: results from the plasma spray coatings are excluded from the chart due to the influence of the much higher coating thickness—70 μm compared to 5 μm); and

FIG. 18 shows the cell density measurements indicating the degree of osteoblast adhesion. The multi-layer films displayed a statistically significant increase in cell density compared to the other two films and the control Ti.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

In accordance with the method of the subject invention, a substrate is coated with calcium phosphate loaded with a therapeutic agent. The crystallinity of the calcium phosphate is controlled to control the release rate of the therapeutic agent. Typically, the loaded calcium phosphate is deposited onto the substrate by an APD process. Ion beam sputtering, electrophoretic deposition, ion beam assisted deposition, and other methods, however, may be used. Typically a therapeutic substance such as a bone morphogenic protein is mixed in solution with calcium phosphate in powder form. Or, the calcium phosphate can be precipitated from a solution including the therapeutic substance.

A typical thickness of the loaded calcium phosphate coating is between 0.1 to 30 μm. In one example, when an APD process is used, particles of loaded calcium phosphate between 0.001 to 200 μm are entrained in a gas jet at 50-220 psi and then directed to the surface of an implant at a distance of typically 0.5-2 inches.

The subject invention represents a process for deposition of a coating including calcium phosphate (CaP) which has been loaded with one or more therapeutic substances. The coating is engineered to release the embedded substance at a controlled rate usually for a local therapeutic effect. The deposition process preferably occurs at temperatures less than 200° C., preferably even at room temperature, which allows the source material to maintain its original size, chemistry and phase composition. Inert gas, such as nitrogen, may be used in the application of the coating but is not incorporated into the coating. In other words, the composition of the source material is exactly what ends up in the coating.

Various nozzle tips have been tested and found to variably affect the APD process and resulting coating properties. The CaP particles are accelerated to such a speed that when they impact the substrate, the particles imbed themselves into the substrate and form a layer of CaP. The coating's adherence and coherence depends on the source material, the substrate material, and various processing parameters, such as pressure, the angle of incidence and the distance from the nozzle to the sample. See also U.S. Pat. Nos. 5,302,414; 6,502,767; 4,968,540 and U.S. Patent Publication No. 2005/0169964A1 all incorporated herein by this reference.

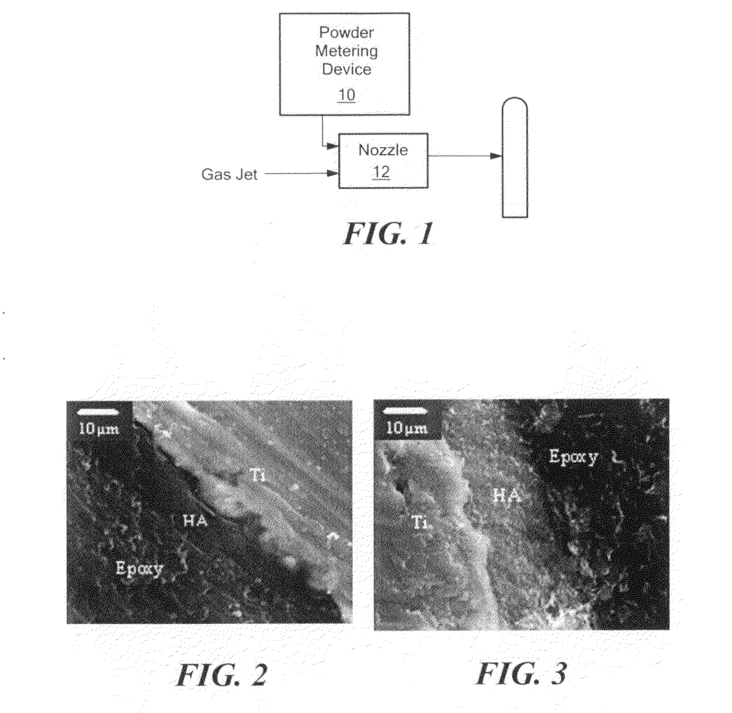

The coating media (CaP powder) is placed in a reservoir 10, FIG. 1 which is then sealed and connected to a control unit. Deposition takes place inside a glovebox, which is under negative air pressure to prevent CaP from escaping into the room. CaP powder is drawn through the control unit and exits through nozzle 12 at a very high acceleration rate. The pressure and deposition rate can be controlled. The deposition stream can also be controlled by the size and geometry of the nozzle, the distance from nozzle to the substrate, and the deposition angle. Relative motion can be provided between the nozzle and the substrate.

The therapeutic substance, i.e., drug, protein, is incorporated into the source material prior to the coating process to promote even distribution of the therapeutic substance throughout the coating. The distribution of the drug(s) have an influence on the release kinetics of the coating.

Another influence on the release kinetics is the crystalline composition of the calcium phosphate. By controlling the crystallinity of the source material and coating, the dissolution rate of the calcium phosphate (scaffold holding the drug in place) can be controlled. Phase compositions that could be utilized in this coating are 100% crystalline hydroxyapatite (HA), highly crystalline HA (80-90%), tricalcium phosphate (TCP), beta tricalcium phosphate (β-TCP), silica calcium phosphate, and amorphous calcium phosphate (ACP). The coating could also be composed of one or more of these calcium phosphate phases. The various phases could be applied all at once or they could be deposited in distinct layers to form a graded coating. The graded coating would provide a method for further controlling the coating response to the environment and the body's response to the coating and implant.

A calcium phosphate-based coating with a therapeutic substance incorporated is advantageous because most drug-eluting coatings on the market today are polymer based. Polymers can elicit an inflammatory response from the body whereas calcium phosphate is a naturally occurring substance in the body and would therefore prevent any foreign body response.

The new drug-eluting coatings can be used for a variety of implanted medical devices and applications including cardiovascular stents and bone-contacting implants.

### Example 1

APD process can accelerate particles to sonic or supersonic state. Using this process, particles have been deposited on a variety of substrates including Ti SS, CoCr, and the like.

The process gas is introduced through a gas control module to a manifold system containing Nitrogen gas to a powder-metering device. The high-pressure gas is introduced into the nozzle; the gas accelerates to sonic velocity in the throat region of the nozzle. The flow then becomes sonic as it expands in the diverging section of the nozzle. See FIG. 1. Typical gas jet parameters for the process are summarized in Table 1:

TABLE 1

| Operation Gas | Air, nitrogen, helium and mixture | |
|---|---|---|
| Jet Internal Pressure | 50 to 200 | psi |
| Jet Temperature | 20 to 30° | C. |
| Spray Distance | 0.5 to 2 | inches |
| Particle size | .001 to 100 | μm |

As shown in Table 1, process gases include nitrogen, helium, air, and mixtures of these gases. Nitrogen is a favored process gas because it can be used to spray some materials without promoting oxidation.

The coating thickness of one coated titanium sample was approximately 10 μm. (See FIGS. 2-3). The Epoxy shown is used in shear and tensile strength testing.

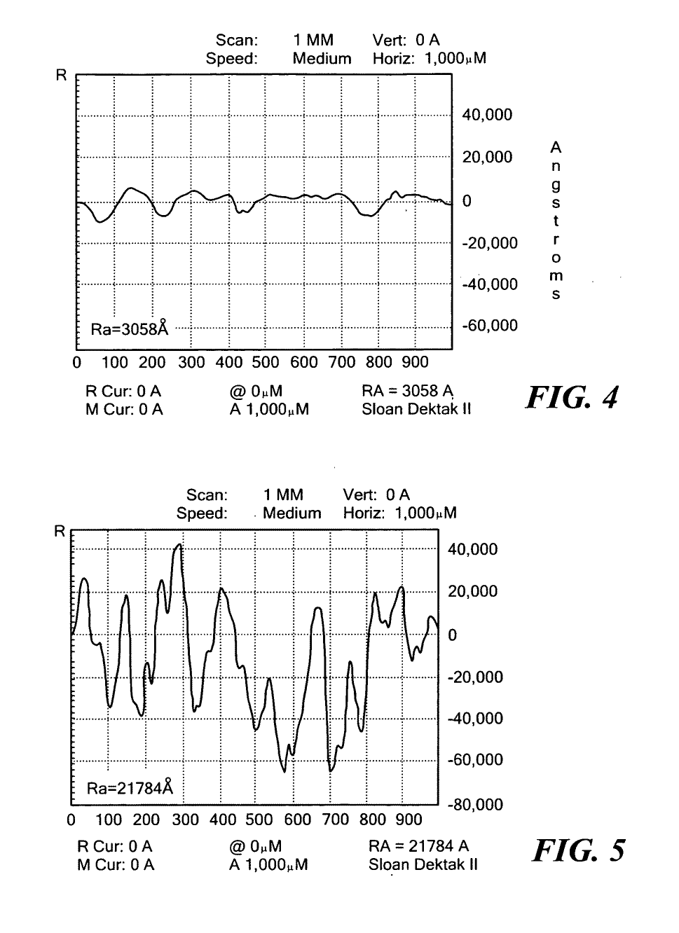

Surface roughness was evaluated for a HA coating deposited on polished titanium samples. Results of surface profilometry evaluations are shown in FIGS. 4-5. FIG. 4 is a profilometry scan of uncoated substrates and FIG. 5 is a scan of a coated substrate. The X-axis units are micrometers; the Y-axis units are Angstroms. The position of origin on both axes is arbitrary. As shown, the surface roughness increases from 0.3 to 2.0 microns Rₐ. The increased roughness is primarily a consequence of the HA coating, and not increased surface roughness induced by the physical bombardment process. Separate experiments demonstrated that stripping the coating away with hydrochloric acid yields the same, pre-coated roughness levels of approximately 0.3 microns.

The coating bond strength was measured according to ASTM Standard F 1147-99, “Standard Test Method for Tension Testing of Calcium Phosphate and Metallic Coatings.” The substrate was Ti-6Al-4V coupons with a dimension of 1.0 inch in diameter and 0.25 inch in thickness. The face of each uncoated coupon was bead blasted with #30 alumina granules before each bond strength test. The adhesive used with the calcium phosphate coating was FM 1000 having a thickness of 0.25 mm. A constant load was applied between the HA coated specimen and the opposition coupon, using a calibrated high temperature spring to apply a stress of 0.138 MPa, (20 psig) during the 2 to 3-hour curing process at 176° C. The bond strength test was performed using a standard tensile test machine with a constant crosshead speed of 0.25 cm/min. The fracture load and fracture surface was recorded for six samples of each HA coating composition to obtain average bond strength and standard deviation. These results were compared to commercially available plasma sprayed HA coatings.

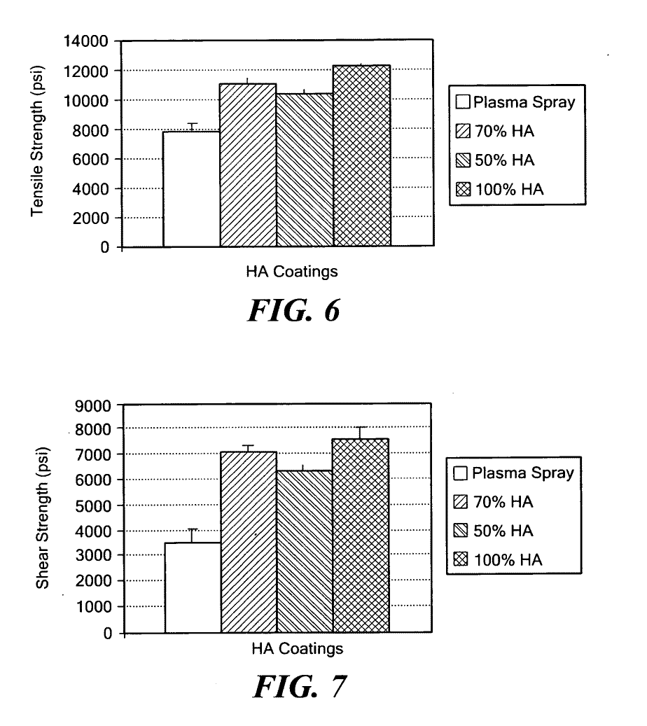

FIG. 6 shows the tensile strength on the different HA coatings per the ASTM standard compared to plasma sprayed HA. This results show that coatings applied in accordance with the process discussed above have better adhesion compared to available commercial plasma sprayed HA.

The shear bond strength of the coating-Ti interface was measured following ASTM F 1658-95, “Standard Test Method for Shear Testing of Calcium Phosphate Coatings.” FM 1000 Adhesive Film (American Cyanamid, N.J.) (with a thickness of 0.25 mm) was used. Six coated Ti6Al4V specimens (cross sectional area of 2.84 cm²) from each HA treatment type were compared to uncoated Ti alloy samples. The bond was achieved at 176° C. for 2-3 hours and at a constant stress of 0.138 MPa using a calibrated high temperature spring. The cured samples were then tested using an Instron pull tester, at a uniform cross-head speed of 0.25 cm/min. The shear strength was calculated using the following formula:

S=Pₘₐₓ/A (1)

where S is the shear strength (MPa), Pₘₐₓ is the maximum load in the test (N), and A is the cross sectional area of the bonded area (cm²).

These results were compared to commercially available plasma sprayed HA coatings. FIG. 7 shows the shear strength of the different HA coatings per the ASTM standard compared to plasma sprayed HA. The results show that the inventive coatings have better adhesion compared to available commercial plasma sprayed HA coatings.

Thus, the preferred coating has tensile strength greater than 8000 psi, a shear strength greater than 5000 psi, and a thickness of 1-20 μm.

To produce the coating, in one example, a therapeutic substance in solution is mixed with the calcium phosphate powder prior to deposition. Therefore, the therapeutic compound is present at all levels of the coating, providing another parameter for controlling the release of the compound.

Another method for incorporating the therapeutic substance into the calcium phosphate (CaP) material is to precipitate the calcium phosphate from a solution containing the therapeutic substance. By using this method, the therapeutic substance becomes embedded within calcium phosphate agglomerates. Additionally the therapeutic substance adsorbs to the surface of the calcium phosphate agglomerates. This method may be used for materials that are prepared at a temperature less than 100° C.

### Example 2

Experiments were conducted to test the release rate of BMP from various formulations of calcium phosphate. The results have uniquely shown that the release rate is dependent on the crystallinity of the calcium phosphate material. Various formulations of calcium phosphate with BMP were coated on commercially pure (CP) titanium (Ti) coupons (1 cm×1 cm) using the method described above. To measure the release rate, the coated substrates were placed in 12 well plates with cell culture medium (DMEM supplemented with 10% FBS (does not contain BMP); Hyclone) and cultured at 37° C. in 95/5% air/CO₂ for up to 21 days. Once a day for 21 days, a small amount (5 microliters) of the supernatant solution was removed from each well and the presence of the imbedded proteins were determined by an ELISA assay with antibodies specific to BMP (Biochem). In this manner, the release rates of BMP per each substrate coating type were determined. Experiments were run in triplicate and repeated at three different times.

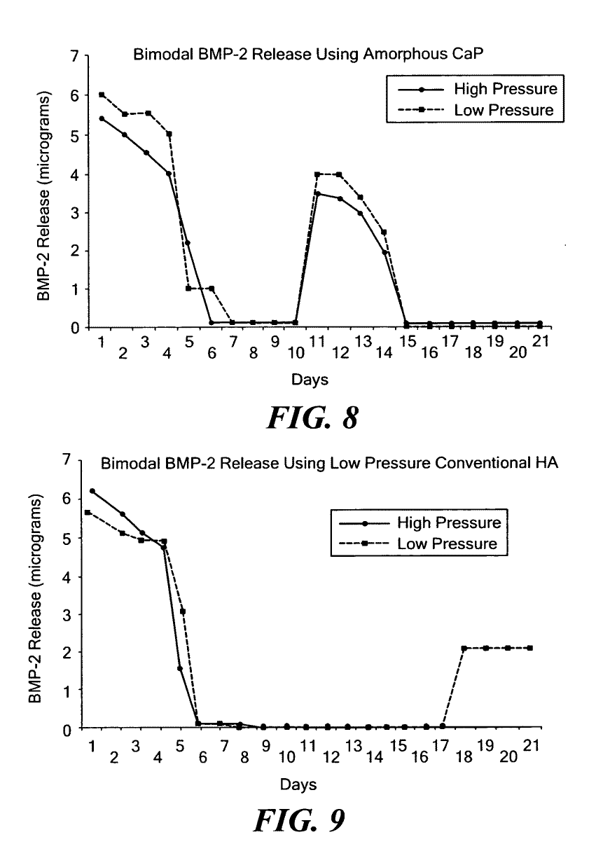

It was found that nano-amorphous CaP with BMP exhibited a bimodal release profile as seen in FIG. 8. FIG. 8 shows the BMP-2 release profile in micrograms over time using amorphous CaP and shows a bimodal release from day 1 to day 7 and day 10 to day 15. The different lines designate different pressures used during the deposition process. There is an immediate release of BMP from the coating that takes place until days 5 or 6. There is no release of BMP from day 6 or 7 to day 10. There is another release of BMP during days 11-15. Finally, there is no BMP present in the solution from day 15 until the end of the testing at day 21. The two different lines in the graph designate two different pressures that were used to deposit the coating, low and high pressure. It was found that the pressure used for deposition affected the density and release rates of the coatings and was therefore used as another parameter to control release rates of the coatings.

### Example 3

In another example, microcrystalline HA was loaded with BMP and then coated onto CP—Ti substrates. These samples were analyzed as described above for release rate. FIG. 9 shows BMP-2 release profile in micrograms over time using microcrystalline HA, exhibiting a bimodal release from day 1 to day 8 and day 17 through day 21 (end of assay). The coating was still present at the end of the assay so BMP release could continue for an unknown period of time. The different lines designate different pressures used during the deposition process. The bimodal release profile shown here is similar to the nano-amorphous CaP/BMP coating. However, one difference is the timing of the second release. The microcrystalline HA/BMP coating releases BMP from day 17 through the end of the test at 21 days.

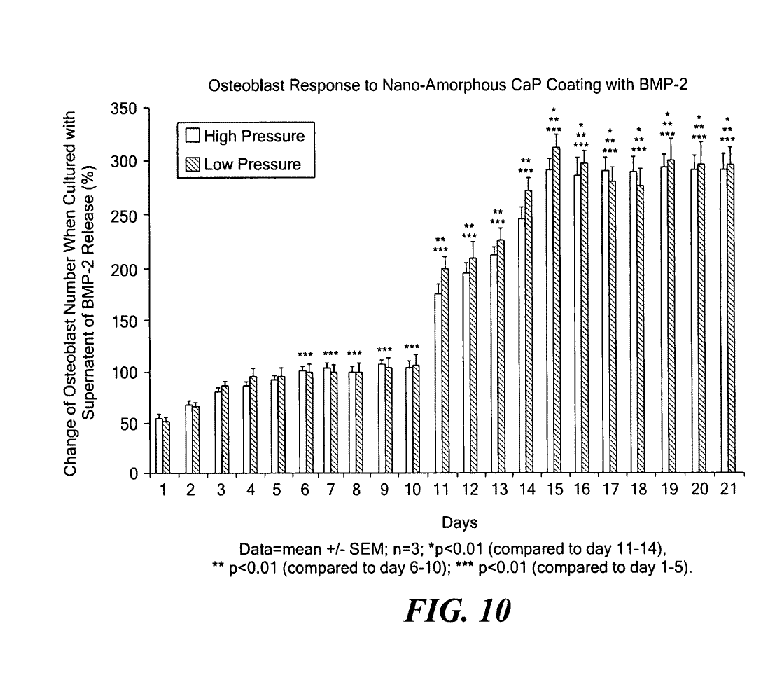

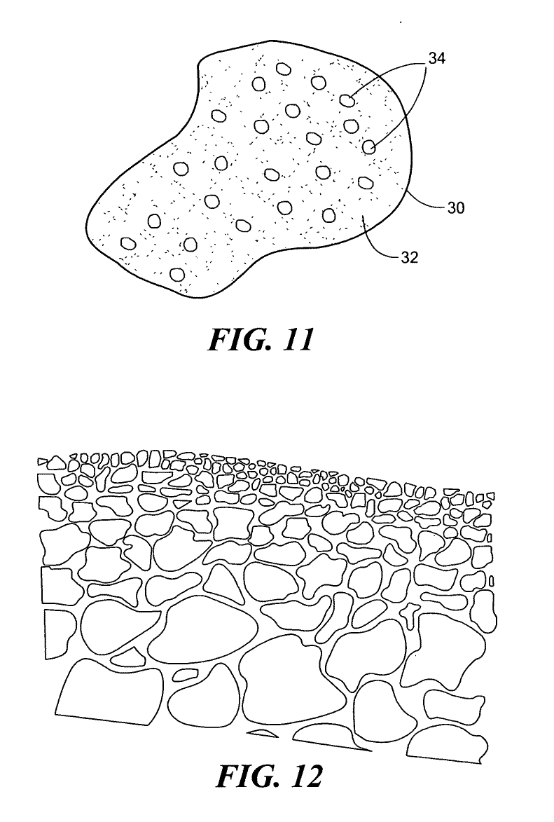

In order to verify that the BMP-2 remained active after the deposition process, coated and uncoated samples were analyzed for cell activity. It is known that BMP causes osteoblasts to proliferate and induces bone formation. To evaluate cell proliferation, human osteoblasts were seeded (3500 cell/cm²) onto glass and were cultured in the presence of the supernatant of the coatings placed in cell culture media for up to 21 days. Cell preparations were examined using a fluorescence microscope with cell density (cells per unit surface area) determined by averaging the number of cells in five random fields. Results of these cell counts were compared to controls (osteoblasts cultured without any supernatant). FIG. 10 shows the percent change in osteoblast number when cultured with supernatant of BMP-2 that has been released from nano-amorphous CaP coating over 21 days. The sharp increase in osteoblast number at day 11 directly correlates with the second release of BMP-2 from the nano-amorphous CaP coating.

FIG. 10 shows calcium phosphate particle 30 loaded with a therapeutic substance 32 amongst calcium phosphate powder grains 34 in accordance with the subject invention. The calcium phosphate powder grains may be amorphous calcium phosphate, fluorapatite, hydroxyapatite, tetracalcium phosphate, tricalcium phosphate-alpha, tricalcium phosphate-beta, biphasic calcium phosphate, silica calcium phosphate, and/or multiphasic calcium phosphate. The therapeutic substance 32 may be a bone morphogenic compound, an antibiotic, or another therapeutic substance or a combination of substances as are known in the art. The volume by weight of calcium phosphate to the therapeutic substance may be equal or of other percentages. The typical calcium phosphate grain 34 is between 10 nm and 10 microns. The crystallinity of the calcium phosphate grains can change. In one example, the agglomerated grains of hydroxyapatite are nano-crystalline in structure. In another example, they are micro-crystalline in structure. A certain percentage of particle 30 can include calcium phosphate in an amorphous state and a certain percentage of calcium phosphate in a crystalline state. Or, certain loaded particles could vary such that a certain percentage of the powder particles of loaded calcium phosphate have one characteristic (nanocrystalline in structure, for example) and they are mixed with a certain percentage of powder particles 30 of loaded calcium phosphate having a different characteristics (amorphous calcium phosphate, for example).

Calcium phosphate or hydroxyapatite (HA) thin films can be deposited that control the osseointegration response to dental implants via microstructural grading. Above, we showed that controlling crystallinity of calcium phosphate (CaP) thin films can enhance device fixation compared to conventional plasma spray HA coatings. Physical vapor deposition (PVD) coatings have been developed where crystallinity changes with film thickness. As shown in FIG. 12, more crystalline regions near the coating/substrate interface transition to very fine-grained, near-amorphous HA crystals at the surface of the film. The finer grained regions of the film will resorb quickly yielding rapid bone growth during early stages of osseointegration, whereas more crystalline regions deeper in the film will resorb more slowly leading to a controlled and sustained long-term osseointegration response. Thin CaP coatings with graded crystallinity provide both more rapid stimulation of host bone cell response and better long-term osseointegration leading to stronger bone fixation.

Ion beam assisted deposition (IBAD) is an advanced thin film deposition technique that combines evaporation with concurrent ion beam bombardment in a high vacuum environment. The ion bombardment enhances adhesion and improves film density. Several investigators have studied formation of HA films by IBAD. These films are always amorphous in the as-deposited state and require subsequent annealing at temperatures exceeding 500° C. to crystallize them. Lowering the crystallization temperature of IBAD films from 500° C. to 400° C. can be accomplished by depositing and annealing the films in humidified environments. Researchers have determined that, because crystallization of HA from the amorphous phase is a hydroxyl-diffusion controlled process, the enriched hydroxyl content in the films decreases the activation energy for nucleation during deposition. Furthermore, the researchers speculate that annealing in a humidified environment decreases the activation energy for diffusion, raising the diffusion coefficient of the hydroxyl ions.

Others have investigated the coating/substrate interface of IBAD HA films deposited on titanium substrates. It was found that the IBAD process creates a gradient region that transitions from titanium to titanium-phosphate to HA. This gradient region significantly increases adhesion of the HA coating. It was also noted that the crystallization of IBAD HA thin films during post-deposition annealing depended on the intensity of the ion beam during deposition. Lower degrees of ion bombardment produced films with mixtures of HA and TCP, while higher bombardment levels resulted in pure crystalline HA.

### Example 4

A stable process was developed for vacuum depositing Ca—P films via IBAD using electron beam evaporation. System tooling and fixturing for coating three-dimensional objects in the vacuum chamber (with applied heating) was also designed and constructed.

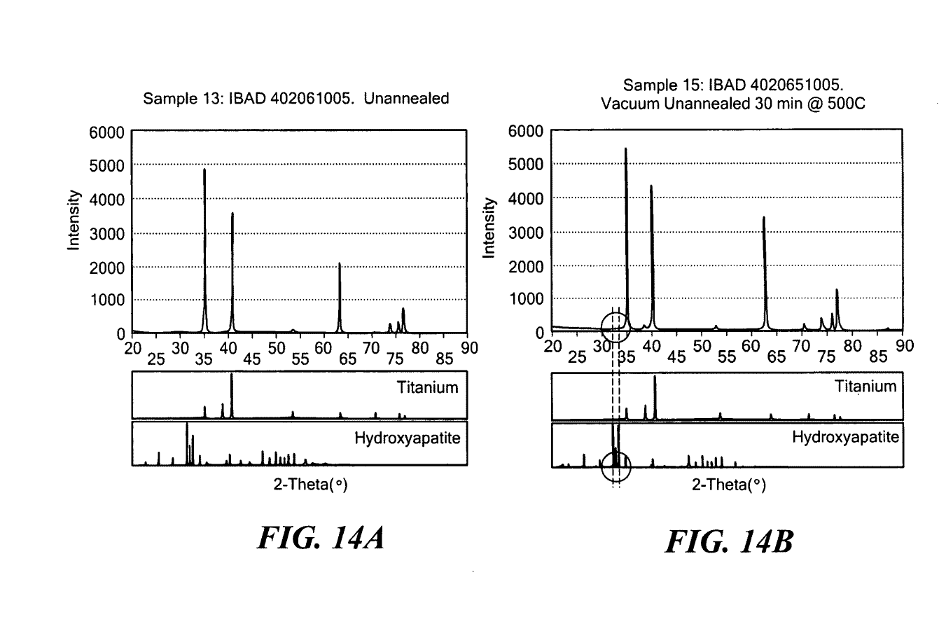

Thin IBAD HA coatings were deposited and the crystallinity evaluated before and after annealing. In the as-deposited state the coatings are amorphous, consistent with findings of other investigators. Film crystallinity was assessed via x-ray diffraction (XRD). A representative spectrum for an IBAD deposited Ca—P film (on a commercially pure titanium substrate) is shown in FIG. 14A. Spectra for titanium and for crystalline hydroxyapatite are shown for reference. The spectrum for the IBAD film appears essentially the same as the titanium substrate, indicating that the film has minimal crystalline content, or that crystallites are very small. Samples were also annealed at 500° C. in air or in vacuum for either 30 or 120 minutes. Subsequent to annealing, samples were evaluated using XRD. All annealed samples contained crystalline HA. Neither annealing time nor environment (air vs. vacuum) affected the results. A typical spectrum is shown in FIG. 14B. Note the presence of the 3 peaks corresponding to crystalline HA between 32 and 34 degrees 2-theta. Although the titanium peaks continue to dominate the spectrum, these lines are clear evidence of HA crystallization in the annealed IBAD films.

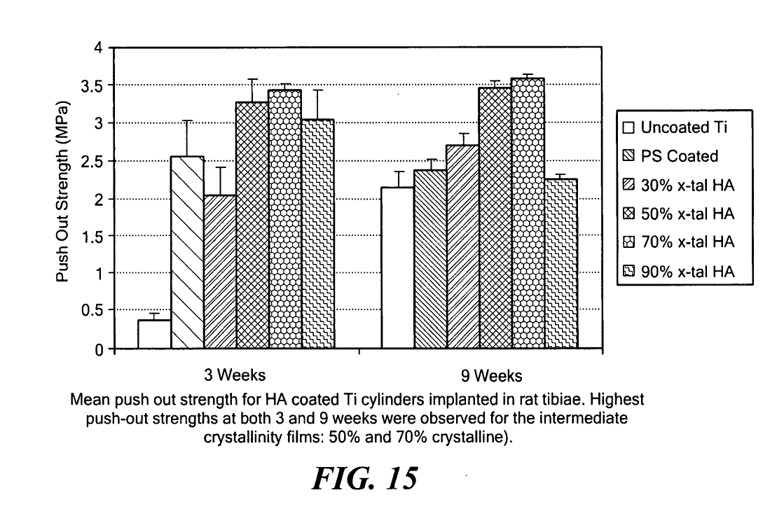

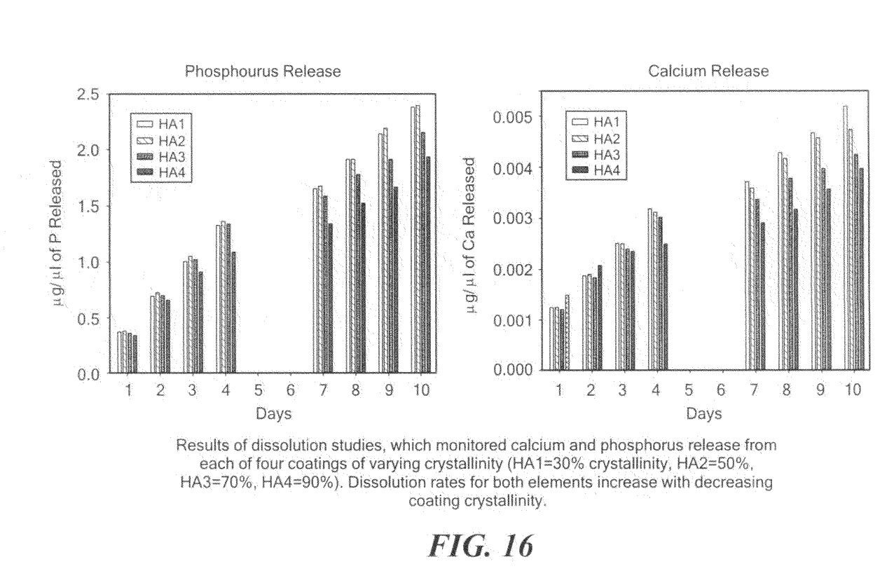

The push-out strength and bone contact length of HA coated implants in rat tibiae was investigated. The objective of these studies was to determine the optimal coating crystallinity for thin HA films. Results, shown below in FIG. 15, demonstrate that films of intermediate crystallinity (50-70%) provide superior performance compared to films of higher or lower crystallinity, and that thin films (2-5 μm) within this range of crystallinity are superior to conventional plasma spray HA coatings.

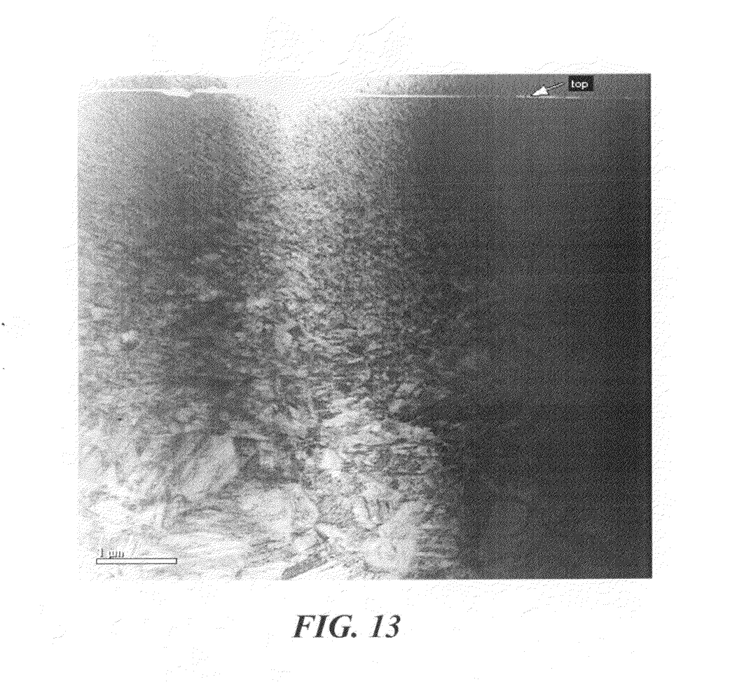

As part of these studies, rates of Ca and P dissolution from the varying crystallinity coatings was assessed. Results showed a direct relationship between decreasing coating crystallinity and increasing dissolution of both elements (FIG. 13). These studies show that crystallinity of calcium phosphate thin films strongly influences bone response, likely due, at least in part, to the different release rates of constituent calcium and phosphorus.

### Example 5

Multi-layer calcium phosphate thin films were developed, consisting of crystalline base layers deposited by an APD process and amorphous top layers deposited by IBAD. These films were evaluated in both cell culture and small animal investigations. Both of these studies showed that the multi-layer films provide superior performance to similarly deposited single-layer films A 2-3 μm thick layer of 100% crystalline hydroxyapatite was deposited using an APD process. On top of that layer, a 0.5 μm layer of IBAD CaP was deposited. This coating was deposited on a series of flat substrates, used for cell culture studies, and cylinders, used for the rat implantation experiments. Results of these studies are discussed below.

The implant samples were cylinders machined from Grade 2 CP titanium stock (President Titanium, Hanson, Mass.). Before coating, they were ultrasonically cleaned in consecutive 10-minute baths of acetone and ethanol. The samples were then ultrasonically rinsed in de-ionized water and dried in a dust-free environment.

The titanium cylinders were APD/IBAD coated at Spire after surface cleaning. All coated samples and uncoated controls were then provided for rat implantation.

Forty-five male Sprague-Dawley rats, about 12 weeks old and weighing about 250-300 grams each, were used. Each animal received 2 implants in the left tibia. Eighteen implants were used for each test condition (uncoated Ti, plasma-spray coated Ti, IBAD-only CaP, 100% crystalline HA (SPA), and multi-layer SPA crystalline HA/IBAD CaP). Nine were evaluated at 3 weeks and the other nine at 9 weeks. At each time point, 6 of the 9 implants were used to assess push-out strength, and the other 3 used for histology. Thus, there were a total of 5 conditions×9 implants per time point×2 time points=90 implants, with 2 implants per rat. All animal experiments were in compliance with NIH publication #86-23, Guide for the Care and Use of Laboratory Animals. Appropriate considerations were given to all policies, standards, and guidelines governing the proper use, care, handling, and treatment of the animals.

Under anesthesia, the tibial bone of each rat was carefully exposed. After dissection of the periosteum, 2 transcortical holes were formed at intervals of 4 mm by drilling with a slow speed (500 rpm) dental handpiece equipped with a 1.8 mm trephine burr to reach the marrow. Profuse irrigation with physiological saline was maintained throughout the drilling. The cylindrical implants were inserted into each of the surgically-prepared holes by tapping with a mallet until the top of the implant was flush with the cortical bone surface. After 3 and 9 weeks implantation, the animals were euthanized and the implants used for either push-out strength tests or histological evaluation as described above. Significant differences between the bone-implant contact lengths and push-out strengths for implants from the different treatment groups were analyzed using ANOVA, with Sheffe's procedure as the post-hoc test.

To evaluate the interfacial strength of the implants at the bone-implant interface, push-out tests were performed using an Instron Model 1125 (Instron Corp., Canton, Mass.). A total of 6 implants/treatment/time point were used to evaluate the interfacial strength. Significant differences in the interfacial strength between different groups of implants were analyzed using ANOVA. Differences were considered statistically significant at p<0.05.

A total of three implants/treatment/time point were used for histology. Bone-implant contact lengths were measured at the bone-implant interface. Significant differences between the bone-implant contact lengths were analyzed using ANOVA, with Sheffe's procedure as the post hoc test. Differences were considered statistically significant at p<0.05.

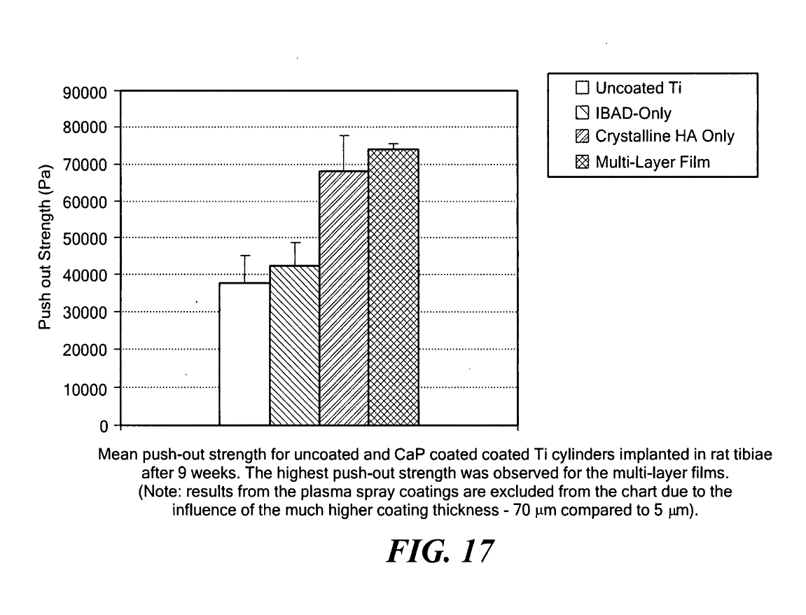

All surgeries were uneventful. However, two of the rats died during the course of the study for unrelated reasons. Because the plasma spray coatings were much thicker, during implantation, the PS-HA coated implants experienced a tighter fit compared to the non-coated titanium and coated test samples. Consequently, plasma spray coated samples exhibited higher push-out strength compared to the other samples. Otherwise, no statistical difference was observed at either 3 or 9 weeks for any of the conditions evaluated. However, at 9 weeks, the combination coating showed higher mean push-out strength compared to either crystalline or IBAD-only CaP coatings (FIG. 17).

Cell culture studies were performed. Three different films were evaluated in these studies for osteoblast adhesion (in addition to the Ti control):
1. Control Ti
2. IBAD-Only
3. Multi-Layer APD/IBAD
4. Multi-Layer APD

The multi-layer APD coating consisted of a 100% ACP layer deposited on top of a 100% crystalline layer, and was tested for comparison purposes.

Rat primary bone-marrow cells were obtained from Wistar rats (2-3 weeks old; Tacomic Farm, N.Y.) using previously developed procedures. Bone marrow cell proliferation on the coated and uncoated substrates were evaluated by seeding cells randomly onto the substrate surface and culturing under standard cell culture conditions for 4 hours. Cell proliferation was assessed by measuring the amount of DNA in papin-digests using Hoeschst 33258 dye (Sigma) and a fluorospectrophotometer (Milton Roy Company, Fluorospectronic) following methods reported in the literature. The number of cells in the experimental samples was determined from a standard curve correlating the amount of DNA per known number of cells (assay sensitive to approximately 1,000). Proliferation is reported as cell density (cells per unit surface area). The rate of proliferation is determined by calculating the slope of the number of cells on each substrate.

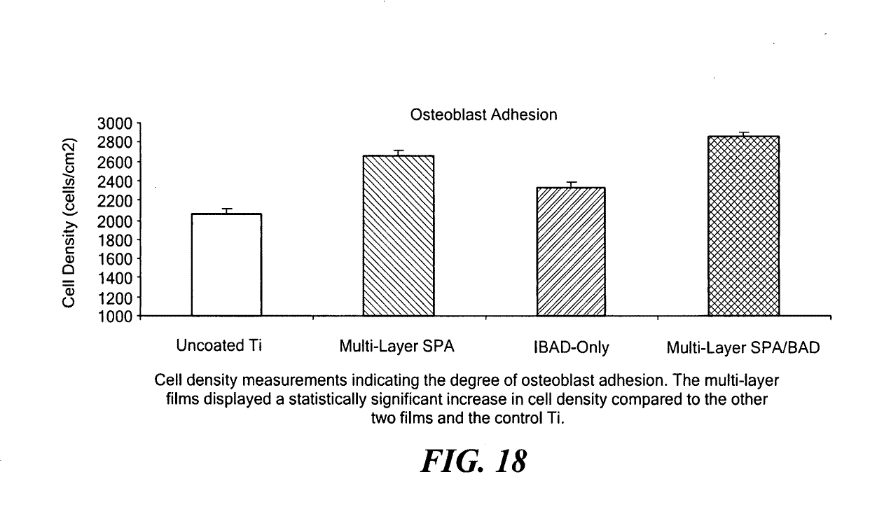

The results (FIG. 18) showed that all coatings had higher osteoblast adhesion/cell density than the control Ti substrate. Additionally, the multi-layer APD/IBAD coating exhibited statistically higher cell densities than any of the other films evaluated.

Films consisting of multiple layers with different crystallinity provide enhanced performance compared to monolithic films with a single crystallinity throughout the film thickness. In both rat implantation experiments and cell culture studies, the multi-layer films provided the highest push-out strength and highest cell densities.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

What is claimed is:

1. A method of coating a substrate, the method comprising:
loading a calcium phosphate substance at a first crystallinity of between 50% and 100% with a therapeutic agent of up to 50% volume by weight;
preparing a calcium phosphate substance at a second crystallinity of between amorphous or near amorphous to 50% crystallinity;
using an accelerated particle deposition process to deposit the loaded calcium phosphate substance at the first higher crystallinity onto at least a portion of the substrate to form a first film having a first thickness; and
depositing the calcium phosphate substance at the second lower crystallinity onto the first film to form a second film thereon having a second thickness thinner than the thickness of the first film.

2. The method of claim 1 in which depositing the second film includes using an accelerated particle deposition process or a physical vapor deposition process.

3. The method of claim 1 in which the calcium phosphate substance is amorphous calcium phosphate, fluorapatite, hydroxyapatite, tetracalcium phosphate, tricalcium phosphate-alpha, tricalcium phosphate-beta, biphasic calcium phosphate, silica calcium phosphate, and/or multiphasic calcium phosphate.

4. The method of claim 2 in which using the accelerated particle deposition process includes particles of loaded calcium phosphate between 0.001 and 200 μm entrained in a gas jet at 50 to 400 psi directed to the surface of the substrate at a distance of 0.5 to 24 inches.

5. The method of claim 1 in which loading includes mixing a therapeutic substance in solution with calcium phosphate in powder form.

6. The method of claim 1 in which loading includes precipitating calcium phosphate from a solution including the therapeutic substance.

7. The method of claim 1 in which the therapeutic substance is a bone morphogenic compound, an antibiotic, an anti-inflammatory agent, an anti-microbial agent, peptide, protein and/or stem cells.

8. The method of claim 1 in which the temperature of the calcium phosphate substance and the therapeutic agent and depositing the same occur at a temperature less than 200° C.

9. A method of coating a substrate, the method comprising:
loading a calcium phosphate substance at a first crystallinity with a therapeutic agent;
depositing the loaded calcium phosphate substance at the first crystallinity onto at least a portion of a substrate;
preparing a calcium phosphate substance at a second, lower crystallinity; and
depositing the calcium phosphate substance at the second crystallinity onto the substrate over the deposited loaded calcium phosphate substance at the first crystallinity to control and sustain a long-term osseointegration response and to control the release rate of the therapeutic substance from the calcium phosphate substance.

10. The method of claim 9 in which depositing the loaded calcium phosphate substance at the first crystallinity includes accelerating particles of the substance to embed the particles in the substrate.

11. The method of claim 9 in which depositing the calcium phosphate substance at the second crystallinity includes a physical vapor deposition process.

12. The method of claim 9 in which the first crystallinity is between 50% and 100%.

13. The method of claim 9 in which the second crystallinity is between amorphous or near amorphous to 50% crystallinity.

14. The method of claim 9 in which depositing the loaded calcium phosphate substance at the first crystallinity includes forming a film having a first thickness and depositing the loaded calcium phosphate substance at the second crystallinity includes forming a second film having a thickness less than the thickness of the first film.

15. A method of coating a substrate, the method comprising:
depositing a calcium phosphate substance at a first crystallinity of between 50% and 100% onto at least a portion of a substrate to form a first film thereon; and
depositing a calcium phosphate substance at a second crystallinity of between amorphous or near amorphous to 50% crystallinity onto the first film to form a second film thereon.

16. The method of claim 15 further including the step of loading the calcium phosphate substance at the first crystallinity with a therapeutic agent of up to 50% volume by weight before deposition on the substrate.

17. The method of claim 15 further including the step of loading the calcium phosphate substance at the second crystallinity with a therapeutic agent of up to 50% volume by weight before deposition on the first film.

18. The method of claim 15 in which depositing the first film includes using an accelerated particle deposition process.

19. The method of claim 15 in which depositing the second film includes using a physical vapor deposition process.

20. The method of claim 15 in which the calcium phosphate substance is amorphous calcium phosphate, fluorapatite, hydroxyapatite, tetracalcium phosphate, tricalcium phosphate-alpha, tricalcium phosphate-beta, biphasic calcium phosphate, silica calcium phosphate, and/or multiphasic calcium phosphate.

21. The method of claim 15 in which the therapeutic substance is a bone morphogenic compound, an antibiotic, an anti-inflammatory agent, an anti-microbial agent, peptide, protein and/or stem cells.

22. The method of claim 15 in which the temperature of the calcium phosphate substance and the therapeutic agent and depositing the same occur at a temperature less than 200° C.

23. A coated substrate comprising:
a calcium phosphate first film having a first thickness and a first crystallinity of between 50% and 100% loaded with a therapeutic agent of up to 50% volume by weight; and
a calcium phosphate second film having a second thickness less than the first thickness on the first film, the second film having a second crystallinity of amorphous or near amorphous to 50% crystallinity.

24. The coated substrate of claim 23 in which the calcium phosphate first film include loaded particles of calcium phosphate embedded in the substrate.

25. An implant with a coating on at least a portion of its surface, the coating comprising:
layers of calcium phosphate at different crystallinity levels wherein at least one of the layers is loaded with a therapeutic compound to control and sustain a long term osseointegration response and to control the release rate of the therapeutic substance.

26. The implant of claim 25 in which the crystallinity of the calcium phosphate decreases as a function of the distance from the implant surface.

27. The implant of claim 25 in which there are at least two layers of calcium phosphate at different crystallinity levels, at least one of which is loaded with a therapeutic compound, a first layer at a first crystallinity of between 50% and 100% and a second layer at a second crystallinity of between amorphous or near amorphous to 50% crystallinity.

28. The implant of claim 25 in which the thickness of each successive calcium phosphate layer decreases as a function of the distance from the implant surface.
